(19) United States
(12) Patent Application Publication
(10) Pub. No.: US 2002/0042078 A1
(43) Pub. Date: Apr. 11, 2002

(54) Method for testing the sterility of, in particular, liquid media

(75) Inventors: Herbert Hermann, Ettlingen (DE); Wolfgang Leis, Eggenstein-Leopolds (DE)

Correspondence Address: Lichti, Lempert, & Lasch, Bergwaldstr. 1, Karlsruhe, D-76227, DE

(73) Assignee: BioChem Labor fur biologische und chemische Analytik GmbH, Karlsruhe (DE)

(21) Appl. No.: 09/968,908

(22) Filed: Oct. 3, 2001

(30) Foreign Application Priority Data
Oct. 11, 2000 (DE) 100 50 135.4

(51) Int. Cl. C12Q 1/68; C12Q 1/22; C12P 19/34

(52) U.S. Cl. 435/6; 435/91.2; 435/31

(57) ABSTRACT

In a method for examining the sterility of in particular liquid media, such as pharmaceutical and/or medical preparations, the microorganisms which may be contained in the media are mechanically concentrated thereby forming a concentrate, and the free nucleic acids which may be in the concentrate are dissociated through the addition of at least one desoxyribonuclease (DNAse). The desoxyribonucleic acid (DNA) and the ribonucleic acid (RNA) of the microorganisms which may be contained in the concentrate are then extracted, thereby simultaneously destroying the cell walls of the microorganisms and of the added DNAse. The DNA and preferably also the RNA are chromatographically isolated and the RNA is transcribed to cDNA through the addition of reverse transcriptase (revertase). The DNA is then concentrated using a polymerase chain reaction (PCR) incorporating primers derived from highly-conserved regions and/or random primers and/or arbitrary primers, and the concentrated DNA is qualitatively evaluated.

This application claims Paris Convention priority of DE 100 50 135.4 filed Oct. 11, 2000 the complete disclosure of which is hereby incorporated by reference.

### BACKGROUND OF THE INVENTION

The invention concerns a method for testing the sterility of, in particular, liquid media, such as pharmaceutical and/or medical preparations.

To examine the sterility of media which meet the highest hygienic standards (e.g. liquid, emulsified or dissolved pharmaceutical and medical preparations, active components, injection and infusion solutions, eye drops or the like, and also surgical implantations), the medium to be examined is conventionally incubated for several days under aseptic conditions under the influence of nutrient media. If the medium becomes visibly cloudy after incubation due to germination, samples are taken and further concentrated for identification. The concentrated germs are subsequently detected using microbiological methods. When germination has been confirmed, the examined sample is identified as not being sterile.

The large amount of time required for incubation and also for further concentration and microbiological determination of the germs, if germination occurs, is disadvantageous, causes high storage costs and requires a substantial amount of space for the samples to be examined. For germ investigations, a large amount of waste is produced. Moreover, the known methods detect only those bacteria, yeast, and fungi etc. for which the respective nutrient media used provides the appropriate growth conditions. Consequently, a considerable number of microorganisms, such as certain bacteria types, in particular mycoplasms, cannot be detected in this fashion.

Polymerase chain reactions (PCR) are conventionally used for selective DNA synthesis of DNA sequences disposed between two short single-strand synthetic oligonucleotides (primers) thereby using DNA polymerase and desoxynucleosintriphosphates (dNTP), desoxyadenintriphosphate (dATP), desoxythymintriphosphate (dTTP), desoxycytosintriphosphate (dCTP) and desoxyguanintriphosphate (dGTP). This genetic engineering method permits selective amplification of certain DNA sections by thermally denaturing the DNA (double-stranded DNA) to be amplified, into a single-strand DNA such that the primers which are complementary at the beginning and end of the DNA region to be amplified, connect to the single-strand DNA (annealing) and are hybridized with this region. The hybridized primers thereby serve as starting points for the heat-resistant DNA polymerase gained e.g. from thermus aquaticus, for the specific synthesis of new double-strands from the desoxynucleosintriphosphates. Conventionally, binding primers are used at the directing strand and opposite strand such that the DNA section disposed between the two primers is doubled. This process can be repeated an arbitrary number of times, wherein each cycle doubles the DNA to be amplified. This permits exponential amplification since each cycle also provides the DNA strands formed during the previous cycles as a matrix.

It is the underlying purpose of the present invention to propose a rapid and inexpensive method for examining the sterility of, in particular, liquid media, such as pharmaceutical and/or medical preparations, which is suitable for detecting substantially all microorganisms and viruses, while thereby preventing false positive results.

### SUMMARY OF THE INVENTION

This object is achieved in accordance with the invention with a method of the above-mentioned type which is characterized by the following steps:

a) concentrating microorganisms which may be contained in the medium, thereby forming a concentrate;

b) dissociation of the free nucleic acids which may be contained in the concentrate by adding at least one desoxyribonuclease (DNAse):

c) extraction of the desoxyribonucleic acid (DNA) and ribonucleic acid (RNA) from the microorganisms which may be contained in the concentrate, thereby simultaneously destroying the cell walls of the microorganisms and the added DNAse;

d) chromatographic isolation of the DNA;

e) concentration of the DNA through polymerase chain reaction (PCR) using primers which are derived from highly-conserved regions and/or random primers and/or arbitrary primers;

f) qualitative determination of the DNA concentrated through PCR.

Compared to prior art, the inventive method advantageously exhibits a considerably reduced examination time of approximately one to two days, since incubation for cultivation of the microorganisms which may be contained in the medium to be examined and, in case of positive results, the subsequent concentration thereof can be omitted. Moreover, the use of suitable, in particular, different primers in the polymerase chain reaction, permits detection of nearly all microorganisms. Due to the high amplification rate of the PCR, the DNA of individual cells is also detected to facilitate the highest possible sensitivity for the method. Dissociation of possible free nucleic acids which are not present in growing microorganisms reliably prevents false positive results through adding at least one DNAse. Finally, the inventive method also detects viruses.

Further features and advantages of the inventive method can be extracted from the following description of a preferred embodiment.

### DESCRIPTION OF THE PREFERRED EMBODIMENT

To examine the sterility of a pharmaceutical solution, the solution is concentrated by e.g. centrifugation, sterile filtration or the like. Microorganisms which may be contained in the solution are concentrated in this fashion and received by a desoxyribonuclease buffer (DNAse buffer). A DNAse, in particular an endonuclease, such as desoxyribonuclease I (DNAse I) is then added. The DNAse treatment exclusively serves for dissociation of free nucleic acids from microorganisms which cannot grow and which may be contained in the concentrate, into mono or dinucleotides. These free extracellular nucleic acids or DNA fragments which are derived e.g. from killed cells, would otherwise produce false positive PCR results, since in PCR all desoxyribonucleic acids contained in the solution are amplified irrespective of whether the microorganisms were alive or dead. The sample to be examined would otherwise therefore be classified as not sterile, even in the event of non-growing microorganisms.

Finally, the DNA and RNA of the microorganisms or viruses which may be present in the concentrate are extracted thereby simultaneously destroying the cell walls of the microorganisms and the added DNAse. For extracting the DNA and the RNA from bacteria which may be contained in the concentrate, lysozymes (peptidoglycan-N-acetylmuramoylhydrolases) and/or lysostaphin (peptidoglycan-endopeptidase) are added. For extracting DNA and RNA from fungi, yeast and other eukaryontes which may be contained in the concentrate, lyticases and/or proteinases, in particular proteinase-K are added, wherein, in particular, proteinase-K eliminates, in an enzymatic fashion, the DNAse previously added for dissociation of free nucleic acids. To extract the entire DNA and RNA of procaryontes and eukaryontes which may be present in the concentrate, an alternative or additional step provides for addition of chaotropic salts, in particular guanidinhydrochloride and/or guanidinisothyiocyanate, and/or carrying out a heat treatment and/or the addition and mechanical agitation of fine particles of an inert material, in particular glass beads.

The DNA extracted in this fashion and preferably also RNA are subsequently chromatographically isolated in a conventional manner e.g. via adsorption chromatography, in particular silicagel adsorption chromatography.

While it is fundamentally possible to only isolate the extracted DNA and use it as starting material for PCR, a preferred embodiment provides that the DNA and also the RNA are isolated (e.g. using silicagel adsorption chromatography) and the RNA is transcribed into complementary DNA (cDNA) by adding at least one reverse transcriptase (revertase). In this fashion, in addition to the DNA, the RNA of the PCR, which is naturally present in much larger amounts in procaryontes and eukaryontes, is also made accessible in the form of cDNA and likewise amplified. The sensitivity of the inventive method is thereby considerably increased.

Finally, the isolated DNA and preferably also the isolated RNA are amplified by means of PCR after being transcribed into cDNA, wherein, in particular, several different primers are used to amplify substantially all of the isolated DNA, irrespective of its origin. In order to identify eukaryontes, such as yeast, fungi and eubacteria, DNA oligonucleotides are used which are complementary to highly-conserved rRNA sequences (ribosomal RNA sequences), in particular primers which are derived from the highly-conserved 16S-rRNA and/or 18S-rRNA sequences. Primers which are derived from highly-conserved snRNA sequences (small nuclear RNA sequences) and/or primers which are derived from the highly-conserved poly-A-tail of mRNA (terminal poly-adenine-group of messenger RNA), preferably oligo(dT)₁₄ primers, can also be used.

In order to detect procaryontes such as bacteria, random primers and/or arbitrary primers are used in addition to primers derived from highly-conserved regions (in this case, in particular, primers complementary to 16S-rRNA) wherein for arbitrary primers a series of arbitrarily selected oligonucleotides with up to ten base pairs are preferably used which can bind to unknown regions of the DNA genome. Substantially completely permutated oligonucleotides, preferably highly-degenerated, are used as random primers and can bind to known and unknown DNA sequences of prokaryontes, such as archebacteria, mycoplasms etc. but also to eukaryontes and viruses. Combination of a plurality of suitable primers ensures amplification of DNA of almost any origin and thus detection of any microbiological trace contamination of the solution to be examined, including viruses.

The polymerase chain reaction (PCR) is suitably carried out in 20 to 50, in particular 30 to 40 sequential cycles, wherein each cycle comprises denaturation of the DNA to be amplified, binding of the primer to the denatured single-strand DNA (annealing) and the synthesis of complementary strands from the desoxynucleosintriphosphates through DNA polymerase.

The qualitative determination of the DNA concentrated through PCR is carried out e.g. in a known fashion through separation thereof using gel electrophoresis, in particular agarose gel electrophoresis, and deposition of at least one fluorescent pigment which can be detected using UV radiation. 3,8-diamino-5-ethyl-6-phenylphenanthridiniumbromide (homidiumbromide, ethidiumbromide) is preferably used as a fluorescent pigment.

The inventive method guarantees rapid, inexpensive and sensitive detection of almost any microorganisms and viruses in liquid or viscous media and in solutions which must meet the highest hygienic standards such as e.g. pharmaceutical and medical preparations. It is also suitable for the examination of the sterility of solid and gaseous media through e.g. extraction thereof and examination of the extract as described above.

We claim:

1. A method for the examination of the sterility of liquid media, pharmaceuticals, and medical preparations, the method comprising the steps of:
a) concentrating microorganisms which may be contained in the medium, thereby forming a concentrate;
b) dissociating free nucleic acids which may be contained in said concentrate by adding at least one desoxyribonuclease (DNAse):
c) extractiing desoxyribonucleic acid (DNA) and ribonucleic acid (RNA) from the microorganisms which may be contained in said concentrate thereby simultaneously destroying cell walls of the microorganisms and said added DNAse;
d) chromatographically isolating said DNA;
e) concentrating said DNA through polymerase chain reaction (PCR) thereby using primers which are derived from at least one of highly-conserved regions, random primers, and arbitrary primers; and
f) qualitatively determining said DNA concentrated through PCR.

2. The method of claim 1, wherein the microorganisms are concentrated using mechanical centrifugation.

3. The method of claim 1, wherein the microorganisms are concentrated via sterile filtration.

4. The method of claim 1, wherein said free nucleic acids contained in said concentrate are dissociated into at least one of mono and dinucleotides by adding at least one endonuclease.

5. The method of claim 4, wherein said endonuclease comprises desoxyribonuclease I (DNAse I).

6. The method of claim 1, wherein at least one of lysozymes, peptidoglycan-N-acetylmuramoylhydrolases, lysostaphin, and peptidoglykan-endopeptidase is added for extracting DNA and RNA from bacteria contained in said concentrate.

7. The method of claim 1, wherein at least one of lyticases, proteinases and proteinase-K is added for extracting DNA and RNA from at least one of fungi, yeast and other eukaryontes contained in said concentrate.

8. The method of claim 1, wherein at least one of chaotropic salts, guanidinhydrochloride and guanidinisothiocyanate are added for extracting DNA and RNA from prokaryontes and eukaryontes contained in said concentrate.

9. The method of claim 1, wherein heat treatment is carried out for extracting DNA and RNA from prokaryontes and eukaryontes contained in said concentrate.

10. The method of claim 1, wherein fine particles of an inert material are added and mechanically agitated for extracting DNA and RNA from prokaryontes and eukaryontes contained in said concentrate.

11. The method of claim 10, wherein said inert material comprises glass beads.

12. The method of claim 1, further comprising chromatographically isolating and transcribing RNA into cDNA by adding at least one reverse transcriptase.

13. The method of claim 1, wherein at least one of DNA and RNA is isolated through one of adsorption chromatography and silicagel adsorption chromatography.

14. The method of claim 1, wherein said primers used in said polymerase chain reaction are derived from a highly-conserved 16S-rRNA sequence.

15. The method of claim 1, wherein said primers used for said polymerase chain reaction are derived from a highly-conserved 18S-rRNA sequence.

16. The method of claim 1, wherein said primers used in said polymerase chain reaction are derived from a highly-conserved snRNA sequence.

17. The method of claim 1, wherein said primers used in said polymerase chain reaction are derived from one of a highly-conserved poly-A-tail of mRNA and Oligo(dT)₁₄ primers.

18. The method of claim 1, wherein arbitrary primers of up to ten base pairs are used for said polymerase chain reaction.

19. The method of claim 1, wherein highly-degenerated, substantially completely permuted random primers are used for said polymerase chain reaction.

20. The method of claim 1, wherein said polymerase chain reaction is carried out in 20 to 50 successive cycles.

21. The method of claim 1, wherein said polymerase chain reaction is carried out in 30 to 40 successive cycles.

22. The method of claim 1, wherein for said qualitative determination of said concentrated DNA, said DNA is dissociated through one of gel electrophoresis and agarose gel electrophoresis and detected using UV radiation after deposition of at least one fluorescent pigment.

23. The method of claim 22, wherein 3,8-diamino-5-ethyl-6-phenyl-phenanthridiniumbromide is used as said fluorescent pigment.
